(19) United States
(12) Patent Application Publication
(10) Pub. No.: US 2010/0183689 A1
(43) Pub. Date: Jul. 22, 2010

(54) Making Method for Titania Nanoparticle

(75) Inventor: Hyung Sup Lim, Ansan (KR)

Correspondence Address: BISHOP & DIEHL, LTD., 1320 TOWER ROAD, SCHAUMBURG, IL, 60173, US

(73) Assignee: Sukgyung AT Co., Ltd., Ansan (KR)

(21) Appl. No.: 12/750,013

(22) Filed: Mar. 30, 2010

Related U.S. Application Data
(63) Continuation-in-part of application No. 12/554,558, filed on Sep. 4, 2009.

(30) Foreign Application Priority Data
Sep. 5, 2008 (KR) 1020080087854

(51) Int. Cl. A61K 8/02; G03G 9/08; A61K 9/14; A61K 33/24

(52) U.S. Cl. 424/401; 430/108.6; 424/489; 424/617; 977/773

(57) ABSTRACT

The present invention relates to a method of manufacturing titania nanoparticles, and specifically to a method of manufacturing titania nanoparticles wherein the particle size is uniform, it is possible to manufacture monodisperse particles without aggregation among particles, a uniform coating can be applied, that is suitable to large-scale production, and that can obtain high-resolution images by maintaining the toner electric charge and electric charge distribution; and the developer included in said titania nanoparticles.

### CROSS-REFERENCE TO RELATED APPLICATIONS

This is a continuation-in-part of U.S. patent application Ser. No. 12/554,558 filed Sep. 4, 2009, pending, which claims priority to Korean application number 10-2008-0087854, filed on Sep. 5, 2008, which is incorporated herein by reference.

### TECHNICAL FIELD

The present invention relates to a method of manufacturing titania nanoparticles, and more specifically, to a method of manufacturing titania nanoparticles wherein the particle size is uniform and the shape is spherical so that it is possible to manufacture uniform, monodisperse spherical particles without aggregation among particles, a uniform coating can be applied, that is suitable to large-scale production for use in many applications.

### BACKGROUND OF THE INVENTION

The dry developers used in electronic photography may be classified as one-part developers that use the toner itself, in which colorants have been dispersed among the terminal resin, and two-part developers wherein a carrier is mixed with the toner.

When copying using these developers, in order to establish a suitable process, the developer must have excellent fluidity, caking resistance, cohesiveness, electrostatic propensity, and cleaning. Inorganic fine particles have been added to the toner in order to increase said fluidity, caking resistance, cohesiveness, and cleaning.

In general, as shown in FIG. 1, external additives typically added to the toner surface have been inorganic particles such as silica (SiO2) and alumina (Al2O3), fluoride microparticles such as vinylidene fluoride and PTFE (polytetrafluoroethylene), and acryl and stylene-acryl resin microparticles manufactured by emulsion polymerization. In FIG. 1, the units are %.

The inorganic particles of the prior art, such as silica, have a diameter of 7-50 nm and are added in order to provide the toner with the fluidity of a powder. Ordinarily, when an external additive with a low particle radius is added to toner, the fluidity is good, but if the silica particle size is too small, it sometimes occurs that the silica separates from the toner surface due to stress applied to the toner, accordingly causing a gradual deterioration in fluidity over time; the size of the external additive particles has a powerful impact on print quality. In addition, because these inorganic particles exist on the far outside surface of the toner, they substantially impact the electrostatic propensity of the toner.

However, hydrophobized silica has strong negative electrostatic propensity, and hydrophobized alumina has strong positive electrostatic propensity, thus having a substantial electrostatic impact on the toner. Accordingly, there is an urgent need for a method of manufacturing monodisperse inorganic particles without aggregation among particles, with nanoscale particles that are also uniform in size and suitable for mass production, and that can be used in high-value-added high-definition toners and next-generation color toners that have a small particle size and require the addition of large quantities of external additives to the toner.

The present invention also relates to sunscreen cosmetics that include specific titanium dioxide powders manufactured in accordance with the present invention that have been rendered hydrophobic.

It is well known that in order to prevent the adverse effects of UV radiation on skin, sunscreen cosmetics that include a UV radiation absorbing agent or UV radiation protection powder are used.

Microparticle titanium dioxide powder has been employed in the past as one such material that effectively protects against the long wavelength UV region and is highly transparent to the visible spectrum.

However, introducing such metal oxide poses problems of cosmetic acceptability. Specifically, the anti-sun products containing them are often in the form of relatively thick emulsions, which are difficult to apply and to spread, heavy, and sticky. In addition, with certain mineral blocking agents, such as titanium dioxide, these defects are accompanied by a whitening effect during spreading on the skin.

Also, in order to obtain high sun protection factors, it is necessary to increase the content of chemical screening agents. For reasons of tolerance, it is sought to avoid using an excessively high level of chemical screening agents, and it is preferred to introduce, alongside or in place of the chemical screening agents, mineral physical blocking agents, in particular metal oxides such as, for example, titanium dioxide, which offers excellent anti-UV properties and very good skin tolerability. However, it is believed that no documents describe employing spherically shaped nanoparticles of titanium dioxide according to the present invention for topical application.

### SUMMARY OF INVENTION

### Problem to be Resolved

The present invention, in seeking to resolve the above-described deficiencies of the prior art, has as its objective a method of manufacturing titania nanoparticles, and specifically to a method of manufacturing titania nanoparticles wherein the particle size is uniform, it is possible to manufacture monodisperse particles without aggregation among particles, a uniform coating can be applied, that is suitable to large-scale production, and that can obtain high-resolution images by maintaining the toner electric charge and electric charge distribution; the nanoparticles manufactured by said method, and the provision of said nanoparticles.

In addition, the present invention has the objective of providing a developer that enables a uniform coating and the obtaining of high-resolution images by maintaining toner charger and charge distribution.

### OTHER INDUSTRIAL APPLICABILITY

Also, the present invention has the objective of providing the spherical nanoparticles titanium dioxide obtained in the manner discussed herein to obtain a sunscreen cosmetic that has a long lasting coverage effect and at the same time has an excellent ease of washability.

It is contemplated that any component which is found in conventional sunscreens may be used in a sunscreen formulation in compositions of the present invention. Furthermore, the monodisperse spherical nanoparticles of titanium dioxide of the present invention may be combined with other metal oxides such as zinc oxide. A person ordinary skill in the art will understand the feasibility of such combinations with other metal oxide. Other components useful in compositions of the invention include an inorganic powder, an organic powder, an inorganic pigment, an organic pigment or a mixture thereof.

Accordingly, the monodisperse spherical shaped nano particle of titanium dioxide comprises components (a) through (d) as follows: (a) a titania precursor, manufactured through a method in which a salt or alkoxide of titania is mixed with a solvent and scanned with microwaves, and (b) a spherical nano particle of titanium hydroxide, produced by adding an alkaline catalyst to the solvent containing titania precursor, and (c) a crystalline spherical titania particle, manufactured through stages of drying and sintering the spherical nanoparticles of titanium hydroxide, and (d) a hydrophobized nano particle of titanium hydroxide.

It is also contemplated that the titania salt or titania alkoxide used to mix with the solvent to manufacture the aforementioned titania precursor comprises one or more selected from among the group comprising titanium oxychloride, titanium chloride, titanium nitrate, titanium sulfate, and C1-C12 titanium alkoxides.

The aforementioned alkaline catalyst is a compound containing an amine or hydroxyl group, or an aqueous solution of same or any other similarly suitable material.

The pH of the alkaline catalyst and the solvent mixture is between 5 and 10.

It is also contemplated that the hydrophobized nano particle mentioned above uses one or more hydrophobization agents from the group comprising hexamethyldisilazane (HMDS), methyltrimethoxysilane (MTMS), dimethyldiethoxysilane (DMDES), and trimethylethoxysilane (TMES), isopropyl triisostearoyl titanate (KR-TTS), isopropyl dimethaacryl isostearoyl titanate (KR-7), isopropyl tri(dodecyl)benzenesulfonyl titanate(KR-9S), isopropyl tri(dioctyl)pyrophosphato titanate (KR-38S), di(cumyl)phenyl oxoethylene titanate (KR-134S), di(dioctyl)pyrophosphate oxoethylene titanate (KR-138S), neopentyl(diallyl)oxy, and tri(dioctyl)pyro-phosphato titanate (LICA-38).

The hydrophobization agent is used at 1 to 20 weight parts with respect to 100 weight parts of crystalline titania nanoparticles (relative to the solid component).

A cosmetic composition comprising a plurality of monodisperse nano particle of titanium dioxide, each said particle having a substantially spherical shape, said plurality of particles does not aggregate upon applying on human skin, wherein said plurality of particles being dispersed within said composition in an amount effective to shield substantially all of said skin over which said composition is applied from hazardous effects of ultraviolet radiation.

In the preparation of the sunscreen cosmetic composition of the invention, the aforementioned particles have a diameter of between 20 to 200 nm. Moreover, a specific surface area is contemplated to be within 20 to 100 m2/g. In addition, the aforementioned particles have a contact angle with respect to water of between 100 to 170°.

In addition, it is widely known that a variety of lithium metal oxides, such as lithium titanium oxides and derivatives thereof have been noted as promising materials for use in negative electrodes for lithium-based batteries. Such lithium metal oxides are useful for the production of lithium-based secondary batteries. Because of the interest in lithium metal oxides, several approaches have been developed for producing lithium metal oxide powders.

Therefore, in another aspect of the present invention pertains to a collection of particles comprising lithium titanium oxide or derivatives thereof, wherein the collection of particles are manufactured according to the methods contemplated herein. More importantly, such collection of nano particles of titanium oxide or derivatives thereof have substantially spherical shape and have a specific surface area between 20 to 100 m2/g and have a diameter of between 20 to 200 nm. Moreover, the collection of spherical nano particles of titanium dioxide of the present invention can be utilized to form a flexible secondary battery.

According to the method of manufacturing titania nanoparticles of the present invention for using same in the making of the secondary lithium battery, the size of particles is uniform so that the particles are in between 20 to 200 nm. As a result, the manufacture of monodisperse particles without aggregation between particles is made possible, and a uniform coating is also made possible. Moreover, lithium batteries based on spherical nano particle titanium dioxides or derivatives thereof can have desirable performance characteristics. In particular, the monodisperse spherical titania have high charging and discharging rates while achieving good cycle-ability. In addition, the nano-particles of titanium dioxide can be used to produce superior, flexible electrodes.

Because of their small size, large surface area and uniformity, the titanium dioxide particles can manifest unique properties and can exhibit surprisingly high energy densities in lithium batteries and improved cycle abilities.

The production of nanoparticles of lithium titanium oxide (Li4Ti5O12) using the monodisperse spherical titanium dioxide as precursor is contemplated in this invention. The titanium oxide particles were produced by (1) mixing a salt or alkoxide of titania with a solvent and scanned with microwaves to synthesize a titania precursor; (2) adding an alkaline catalyst to the solvent containing titania precursor obtained in stage (1) above, so as to produce spherical nanoparticles of titanium hydroxide; (3) forming crystalline spherical titania particles through stages of drying and sintering the titanium hydroxide obtained in step (2) above; and (4) undertaking a step wherein the nanoparticles obtained in step (3) above are hydrophobized.

The embodiments described above are intended to be illustrative and not limiting. Additional embodiments are within the claims below. Although the present invention has been described with reference to preferred embodiments, a person skilled in the art will recognize that changes may be made in form and detail without departing from the spirit and scope of the invention.

### Means of Resolving Problem

In order to attain the above objectives, the present invention provides a method of manufacturing titania nanoparticles comprising: (1) a stage wherein a salt or alkoxide of titania is mixed with a solvent and scanned with microwaves to synthesize a titania precursor; (2) a stage wherein an alkaline catalyst is added to the solvent containing titania precursor obtained in stage (1) above, so as to produce spherical nanoparticles of titanium hydroxide; (3) a stage wherein crystalline spherical titania particles are made through stages of drying and sintering the titanium hydroxide obtained in step (2) above; and (4) a step wherein the nanoparticles obtained in step (3) above are hydrophobized.

In addition, the present invention provides monodisperse spherical titania nanoparticles manufactured by said method.

Further, the present invention provides a developer that includes said spherical titania nanoparticles.

### Effects

According to the method of manufacturing titania nanoparticles of the present invention, the size of particles is uniform, the manufacture of monodisperse particles without aggregation between particles is made possible, a uniform coating is made possible, and images of high resolution suitable for mass production can be obtained by maintaining the charge and charge distribution of the toner; when the toner external additive for developing obtained from said titania nanoparticles is used, a uniform coating is possible, and high-resolution images can be obtained by maintaining the charge and charge distribution of the toner.

In addition, according to the method of manufacturing titania nanoparticles of the present invention, it is possible to provide a sunscreen cosmetic to which hydrophobic compound treated monodisperse nanoparticles of titanium dioxide, having substantially spherical in shape, which give the sunscreen cosmetic excellent long lasting coverage against the ultraviolet radiation.

### BRIEF DESCRIPTION OF THE DRAWINGS

FIG. 1 shows a schematic diagram of toner that contains an external additive

FIG. 2 is a schematic diagram of the titania nanoparticle manufacturing method of the present invention, including a microwave scanning device

FIGS. 3 through 6 are scanning electron microscopy (SEM) photographs of titania nanoparticle manufactured according to the respective practical examples of the present invention.

FIGS. 7 through 10 are photographs of the contact angle with respect to water of the titania nanoparticles manufactured according to the respective practical examples of the present invention.

### DETAILED DESCRIPTION OF THE INVENTION

The following descriptions of detailed embodiments are for exemplifying the principles and advantages of the inventions claimed herein. They are not to be taken in any way as limitations on the scope of the inventions.

The toner described hereinbelow in this specification (“toner”) includes both color toner and black/white toner. In addition, “spherical” refers not solely to a perfect sphere, but includes spheroids with a sphericity of 0.6-1. Sphericity (in the case of a sphere) refers to the ratio of the surface of area of a sphere having the same volume as the actual particle to the surface area of the actual particle.

The method of manufacturing titania nanoparticles of the present invention enables the manufacturing of particles of a uniform size and is appropriate for mass production according to studies of the synthesis process technology for spherical titania nanoparticles, and can resolve the problems in aggregation due to positive or negative charge occurring when using an external toner additive such as the silica or alumina of the prior art, through coating the surface of the monodisperse spherical particle with a hydrophobic substance.

The method of manufacturing titania nanoparticles of the present invention comprises: (1) a stage wherein a salt or alkoxide of titania is mixed with a solvent and scanned with microwaves to synthesize a titania precursor; (2) a stage wherein an alkaline catalyst is added to the solvent containing titania precursor obtained in stage (1) above, so as to produce spherical nanoparticles of titanium hydroxide; (3) a stage wherein crystalline spherical titania particles are made through stages of drying and sintering the titanium hydroxide obtained in step (2) above; and (4) a stage wherein the nanoparticles obtained in step (3) above are hydrophobized.

The individual steps of the method of manufacturing titania nanoparticles of the present invention can be described in detail as follows.

### [Step 1]

The present step involves the making of a spherical titania precursor by first mixing titanium salt or titanium alkoxide with solvent and then scanning with microwaves; the microwaves used have a wavelength of 300-3000 MHz; the solvent is instantly heated by the microwave scanning, and the titania precursor is formed.

It is preferable in this step (1) that the solvent be passed through a reaction tube that is scanned by the microwaves, as depicted in FIG. 2; by way of a specific example, it is possible to adjust the reaction outlet temperature to 70-80° C. by proceeding at a solvent fluid velocity of 300-1500 cc/min in the reaction tube furnished by a microwave scanning deice with a maximum output of 5 kW, having an isolator and a magnetron generating 2450 MHz, and setting the reaction speed pass-through time 10-60 sec.

For said titania salt, for example titanium oxychloride, titanium chloride, titanium nitrate, or titanium sulfate may be used; for said titanium alkoxide, a C1-C12 titanium alkoxide may be used; by way of a specific example, titanium ethoxide, titanium isopropoxide, or titanium butoxide may be used.

Said solvent is not limited to any solvent that can dissolve titanium salt or titanium alkoxide; by way of specific example, water, alcohol or an aqueous solution of alcohol may be used. For the alcohol, it is preferable that a C1-C5 alcohol be used; specific examples include methyl alcohol, ethyl alcohol, propyl alcohol, isopropyl alcohol, and butyl alcohol, either singly or in mixture; it is most preferable that the solvent be an aqueous solution of alcohol containing 30-95 vol % alcohol.

In addition, the concentration of said titania salt or titania alkoxide in the solvent should be 0.1-1 M/liter; a dispersant may be used to prevent aggregation, and said dispersant may be such as HPC, PVA, or PVP; of these, HPC allows the most monodisperse particles to be obtained; the quantity of dispersant used should preferably be 0.1-2 g per liter of the total mixture.

### [Step 2]

In Step (2) of the present invention, an alkaline catalyst is added to the solution containing the titania precursor obtained in step (1) above, to produce titanium hydroxide. Here it is preferable that the pH of the solution be adjusted to the 5-10 range by the addition of said alkali.

Said alkaline catalyst may suitably be a compound containing an amine or hydroxy group, or an aqueous solution thereof; specific examples of this include ammonia, sodium hydroxide, alkyl amines, and mixtures thereof.

### [Step 3]

In step (3) of the present invention, the titania hydroxide obtained in step (2) above is dried and sintered; it is preferable that this drying be performed for 4 to 12 hours at 100-130° C., after preparatory drying for 1 to 3 hours at 50-70° C. In addition, the sintering step involves imparting a crystalline character; it is preferable that sintering be performed for 1 to 4 hours at 600-800° C. so as to acquire a rutile shape.

### [Step 4]

Next, in step (4) of the present invention, the surface of the titania nanoparticles obtained in step (3) above is hydrophobized so as finally to produce titania nanoparticles with a hydrophobized surface.

Said hydrophobization may be performed using an ordinary silane coupling agent or titanium coupling agent; specific examples of a silane couple agent include the hydrophobization agents hexamethyldisilazane (HMDS), methyltrimethoxysilane (MTMS), dimethyldiethoxysilane (DMDES), and trimethylethoxysilane (TMES); specific examples of a titanium coupling agent include the hydrophobization agents isopropyl triisostearoyl titanate (KR-TTS), isopropyl dimethaacryl isostearoyl titanate (KR-7), isopropyl tri(dodecyl)benzenesulfonyl titanate(KR-9S), isopropyl tri(dioctyl)pyrophosphato titanate (KR-38S), di(cumyl)phenyl oxoethylene titanate (KR-134S), di(dioctyl)pyrophosphate oxoethylene titanate (KR-138S), neopentyl(diallyl)oxy, and tri(dioctyl)pyro-phosphato titanate (LICA-38).

Said hydrophobization agent may be used in quantities of 1 to 20 weight parts per 100 weight parts of titania nanoparticles (relative to the solid component).

The titania nanoparticles manufactured according to the present invention described above have a monodisperse, spherical form with nearly identical size; the surfaces of these monodisperse spherical particles is coated with a hydrophobic substance, thereby enabling effective use as a external toner additive.

The size of titania nanoparticles of the present invention, thus manufactured, can be adjusted at will; when used as an external toner additive, the size should be from 30 to 200 nm; as needed, the spheres may have a median diameter of 30 nm, 50 nm, 100 nm, 150 nm, or 200 nm.

In addition, the titania nanoparticles of the present invention show a contact angle of 100° or greater with respect to water. (In the case of the contact angle with water, the measured limit value was 170°, but in theory it could be up to 180°.) If said contact angle with water is less than 100°, hydrophobicity will suffer and when used as an external toner additive, the print quality of the toner may suffer due to either the adsorption of airborne moisture or the formation of aggregates.

In addition, it is preferable that the specific surface area of the titania nanoparticles be between 20 and 100 m2/g. If said specific surface area is less than 20 m2/g, the aggregation of particles may be severe; because this makes it difficult for the final coating of the toner with external additive to be uniform, it may cause a problematic deterioration in toner print quality; if it exceeds 100 m2/g, this indicates that the initial particles will be very small, which also makes the hydrophobic coating of individual particles difficult; this may cause a problematic failure of some areas to print due to the toner surface being completely surrounded even at low quantities.

The titania nanoparticles of the present invention manufactured as above-described may be used as external toner additives, and specifically as external toner additives for electrostatic image development. Said external toner additives may be used separately, or also as two or more types together.

When said titania nanoparticles are used as an external toner additive, the ratio of admixture should preferably be from 0.01 to 20 weight parts with respect to 100 weight parts of toner particles; it is even more preferable that 0.1 to 5 weight parts be used. If the admixture ratio is within said range, sufficient adhesion to the toner particles will occur, and not only will good fluidity be obtained, but there will also be a positive improvement in the electrostatic propensity of the toner particles.

Said titania nanoparticles can simply adhere mechanically to the toner particle surface, or may also be fixed gradually to the surface. In addition, the entire surface of the toner particle may be covered, or a portion may be covered.

Toner for electrostatic image development using titania nanoparticles as an external toner additive as above-described may be used as a single-component developer, but it is also possible to blend this with a carrier and use as a 2-component developer. When used as a 2-component developer, the external toner additive should not be added to the toner particles in advance, but only when the carrier is mixed with the toner particles to carry out the surface coating of the toner particles.

This carrier can be any commonly-known carrier such as iron, and can be mixed according to the mixing ratios that are commonly known.

Alternatively, the conventional shaped (e.g., needle or spindle) titanium dioxide are insufficient in the effect for blocking ultraviolet ray and, furthermore, the feeling to the skin becomes poorer, and the finish becomes powdery. To overcome this, the spherical shaped nano particles of titanium dioxide according to the present invention resides in the formulation into a cosmetic composition having an average size from 30 to 200 nm, the spheres may have a median diameter of 30 nm, 50 nm, 100 nm, 150 nm, or 200 nm.

The cosmetic composition comprising the spherical shaped nano particles of titanium dioxide according to the present invention has a protective effect in a broad UV region of UVB and UVA and can simultaneously prevent erythema and melanism caused due to UV rays. Further, the spherical shaped nano particles of titanium dioxide according to the present invention do not aggregate even if formulated together with titanium dioxide or metal iron oxide used for pigments, so there is no rough feeling in a cosmetic containing the same and a product with a smooth feel is obtained.

It is also contemplated by the inventors that the spherical titania nanoparticles of the invention can also be useful in other applications wherein it is desirable to absorb ultraviolet light such as in cosmetics or personnel care products, within or upon packaging material for food or other materials such as wood coatings, coatings on vinyl and other architectural materials, glass, automotive clearcoats, automotive basecoats that may be subject to degradation by UV light, among many other applications.

Hereinbelow, in order to assist in the understanding of the present invention, preferred embodiments are presented; however, these embodiments merely exemplify the present invention and the scope of the present invention is not limited by the embodiments below.

### Practical Examples

### Embodiment 1

### Practical Example 1

### Manufacture of Spherical Titania Nanoparticles of 30 nm Diameter

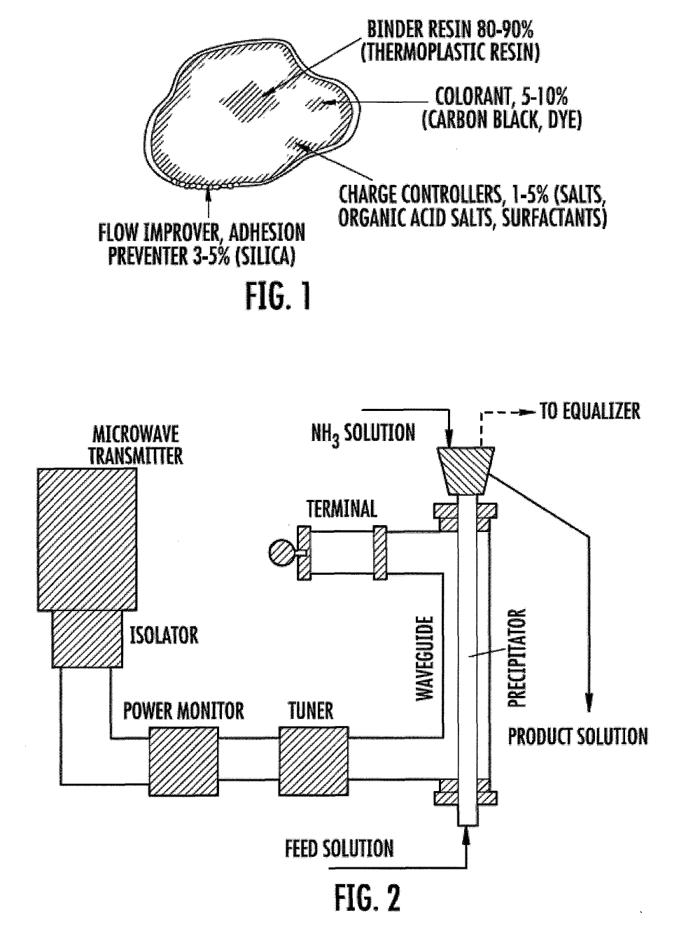

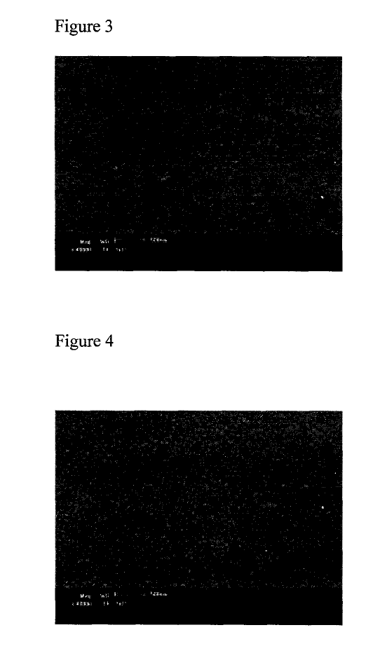

A microwave scanning device (Japan Radio Corporation, JRC: microwave generation device (NJA)) was furnished as shown in FIG. 1 and synthesis was carried out under the conditions of Table 1 below; as a result, spherical titania precursors with an average diameter of 30 nm could be obtained, and after filtering and drying these, heat treatment was performed to yield a powder of titania nanoparticles having a size of 30 nm.

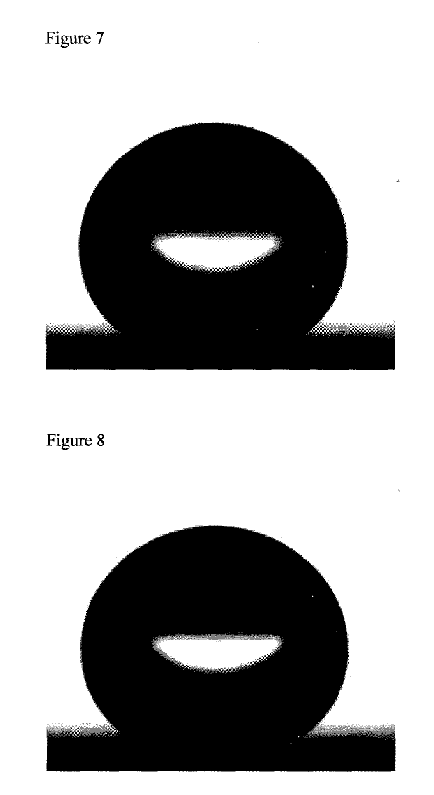

The specific surface area of said powder was measured to be 58 m2/g. Said yielded titania nanoparticles were added to dimethyldiethoxysilane (DIVIDES) at 16.57 weight parts per 100 weight parts, and refluxing and hydrophobization was performed thereon to obtain titania nanoparticles. The contact angle of the surface-treated titania nanoparticles was confirmed by measurement to be at least 150°, as shown in FIG. 7.

### Embodiment 2

### Practical Example 2

### Manufacture of Spherical Titania Nanoparticles of 50 nm Diameter

The microwave scanning device (please indicate manufacturer and product name) used in Practical Example 1 above was employed and synthesis was carried out under the conditions of Table 1 below; as a result, spherical titania precursors with an average diameter of 50 nm could be obtained, and after filtering and drying these, heat treatment was performed to yield a powder of titania nanoparticles having a size of 50 nm, as shown in FIG. 4. The specific surface area of said powder was measured to be 42 m2/g. Said yielded titania nanoparticles were added to dimethyldiethoxysilane (DMDES) at 12 weight parts per 100 weight parts, and refluxing and hydrophobization was performed thereon to obtain titania nanoparticles. The contact angle of the surface-treated titania nanoparticles was confirmed by measurement to be at least 150°, as shown in FIG. 8.

### Embodiment 3

### Practical Example 3

### Manufacture of Spherical Titania Nanoparticles of 100 nm Diameter

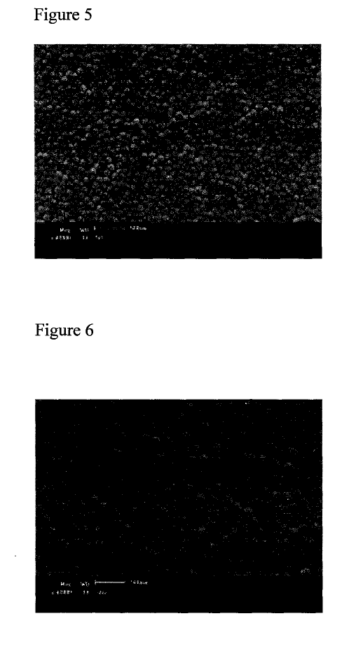

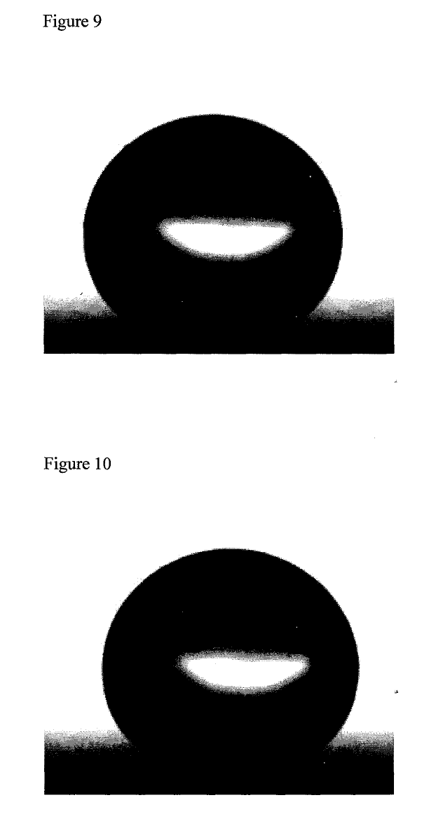

The microwave scanning device (please indicate manufacturer and product name) used in Practical Example 1 above was employed and synthesis was carried out under the conditions of Table 1 below; as a result, spherical titania precursors with an average diameter of 100 nm could be obtained, and after filtering and drying these, heat treatment was performed to yield a powder of titania nanoparticles having a size of 100 nm, as shown in FIG. 5. The specific surface area of said powder was measured to be 25 m2/g. Said yielded titania nanoparticles were added to dimethyldiethoxysilane (DMDES) at 7.14 weight parts per 100 weight parts, and refluxing and hydrophobization was performed thereon to obtain titania nanoparticles. The contact angle of the surface-treated titania nanoparticles was confirmed by measurement to be at least 150°, as shown in FIG. 9.

### Embodiment 4

### Practical Example 4

### Manufacture of Spherical Titania Nanoparticles of 200 nm Diameter

The microwave scanning device (please indicate manufacturer and product name) used in Practical Example 1 above was employed and synthesis was carried out under the conditions of Table 1 below; as a result, spherical titania precursors with an average diameter of 200 nm could be obtained, and after filtering and drying these, heat treatment was performed to yield a powder of titania nanoparticles having a size of 200 nm, as shown in FIG. 5. The specific surface area of said powder was measured to be 17 m2/g. Said yielded titania nanoparticles were added to dimethyldiethoxysilane (DMDES) at 4.86 weight parts per 100 weight parts, and refluxing and hydrophobization was performed thereon to obtain titania nanoparticles. The contact angle of the surface-treated titania nanoparticles was confirmed by measurement to be at least 150°, as shown in FIG. 10.

TABLE 1

| | | Practical | Practical | Practical |
|---|---|---|---|---|
| Operation | Practical Example 1 | Example 2 | Example 3 | Example 4 |
| TiOCl2 | 0.02M | 0.04M | 0.06M | 0.1M |
| concentration | | | | |
| Reaction | 76 | 77 | 78 | 73 |
| outlet | | | | |
| temperature | | | | |
| (° C.) | | | | |
| Flow Rate | 1020 | 910 | 800 | 750 |
| (cc/min) | | | | |
| Res. Time | 12.7 | 14.1 | 16.2 | 17.6 |
| (sec) | | | | |
| pH (NH4OH | 7.57 | 8.87 | 8.43 | 8.20 |
| 1N solution) | | | | |
| Output | ≈30 | ≈50 | ≈100 | ≈200 |
| Sample | | | | |
| Average | | | | |
| diameter | | | | |
| (nm) | | | | |

### Practical Examples 5 through 8

### Manufacture of Toner Mixed with External Additive (Including a Developer Component)

After fusing 4 weight parts of colorant (product name: Carmine 6BC, Smika Color Mfr.) to 96 weight parts of polyester resin with a softening point of 100° C. and a glass transition temperature of 60° C., while kneading and crushing, it was separated to yield toner with an average particle diameter of 7 μm. Toner mixed with external additives was manufactured (Practical Examples 5-8) by mixing 0.3 g each of the titania nanoparticles produced in Practical Examples 1 through 4 above to 10 g of this toner.

In order to verify the performance of the developer of the present invention, the developer produced in Practical Examples 5 through 8 above was used and measured with respect to the quantity of toner used, by the method below; the results thereof are shown in Table 2.

Measurement was performed by

a) a step wherein the weight of the CRU (toner cartridge) was measured before performing the experiment;

(b) a step wherein 5000 prints were made on writing/A4 sized paper;

(c) a step wherein after the completion of 5000 prints, the weight of the CRU was measured; and

(d) a step wherein the consumption of toner per 5000 prints was obtained, and next the amount of toner consumed in print 1 sheet was obtained. By way of a comparison example, a developer manufactured in the same fashion as Practical Example 5, except that none of the above-described titania nanoparticles of the present invention were used, was employed (Comparison Example 1).

TABLE 2

| 1-component | | | | | |
|---|---|---|---|---|---|
| developer | Example 5 | Example 6 | Example 7 | Example 8 | Example 1 |
| Toner usage | 17.7 | 18.3 | 16.9 | 19.6 | 23.8 |
| (mg/pg | | | | | |
| @78/80) | | | | | |

When the developer of Examples 5-8 of the present invention was used, a clear image of high quality was obtained in the prints, and in particular, as is apparent in Table 2, a clear reduction in toner consumption could be observed.

What is claimed is:

1. A monodisperse spherically shaped nano particle of titanium dioxide, comprising components (a) through (c) as follows:
(a) a titania precursor;
(b) a spherical nano particle of titanium hydroxide, wherein said titanium hydroxide is hydrophobic; and
(c) a crystalline spherical titania particle.

2. The titanium dioxide of claim 1, wherein the titania precursor is manufactured through a method in which a salt or alkoxide of titania is mixed with a solvent and scanned with microwaves.

3. The titanium dioxide of claim 1, wherein the spherical nano particle of titanium hydroxide is produced by adding an alkaline catalyst to the solvent containing titania precursor.

4. The titanium dioxide of claim 1, wherein the crystalline spherical titania particle is manufactured through stages of drying and sintering the spherical nanoparticles of titanium hydroxide.

5. The titanium dioxide of claim 1, wherein the titania salt or titania alkoxide comprises one or more selected from among the group comprising titanium oxychloride, titanium chloride, titanium nitrate, titanium sulfate, and C1-C12 titanium alkoxides.

6. The titanium dioxide of claim 1, wherein the alkaline catalyst is a compound containing an amine or hydroxy group, or an aqueous solution of same.

7. The titanium dioxide of claim 1, wherein the pH of the alkaline catalyst and the solvent mixture is between 5 and 10.

8. The titanium dioxide of claim 1, wherein the drying stage takes place over 4 to 12 hours at 100-130° C. after preparatory drying at 50-70° C. over 1 to 3 hours.

9. The titanium dioxide of claim 1, wherein the sintering stage takes place over 1 to 4 hours at 600-800° C.

10. The titanium dioxide of claim 1, wherein the hydrophobized nano particle uses one or more hydrophobization agents from the group comprising hexamethyldisilazane (HMDS), methyltrimethoxysilane (MTMS), dimethyldiethoxysilane (DMDES), and trimethylethoxysilane (TMES), isopropyl triisostearoyl titanate (KR-TTS), isopropyl dimethaacryl isostearoyl titanate (KR-7), isopropyl tri(dodecyl)benzenesulfonyl titanate(KR-9S), isopropyl tri(dioctyl)pyrophosphato titanate (KR-38S), di(cumyl)phenyl oxoethylene titanate (KR-134S), di(dioctyl)pyrophosphate oxoethylene titanate (KR-138S), neopentyl(diallyl)oxy, and tri(dioctyl)pyro-phosphato titanate (LICA-38).

11. The titanium dioxide of claim 1, wherein the hydrophobization agent is used at 1 to 20 weight parts with respect to 100 weight parts of crystalline titania nanoparticles (relative to the solid component).

12. A cosmetic composition comprising a plurality of monodisperse nano particle of titanium dioxide, each said particle having a substantially spherical shape, wherein said plurality of particles being dispersed within said composition in an amount effective to shield substantially all of said skin over which said composition is applied from hazardous effects of ultraviolet radiation.

13. The composition of claim 12, wherein said particles have a diameter of between 20 to 200 nm.

14. The composition of claim 12, wherein said particles have a specific surface area of between 20 to 100 m2/g.

15. The composition of claim 12, wherein said particles have a contact angle with respect to water of between 100 to 170°.

16. The composition of claim 12, wherein an inorganic powder, an organic powder, an inorganic pigment, an organic pigment or a mixture thereof is further formulated therein.
